(19) United States
(12) Patent Application Publication
(10) Pub. No.: US 2025/0275984 A1
(43) Pub. Date: Sep. 4, 2025

(54) ORAL SURGERY DRUG

(71) Applicant: PSKDDS, LLC, Indianapolis, IN (US)

(72) Inventor: Patrick Kelly, Indianapolis, IN (US)

(21) Appl. No.: 19/066,847

(22) Filed: Feb. 28, 2025

Related U.S. Application Data
(60) Provisional application No. 63/559,542, filed on Feb. 29, 2024.

(51) Int. Cl. A61K 31/573; A61K 9/00; A61K 31/407; A61P 23/02

(52) U.S. Cl. CPC A61K 31/573; A61K 9/0019; A61K 31/407; A61P 23/02

(57) ABSTRACT

An injectable composition for use in invasive oral procedures, including Dexamethasone and ketorolac tromethamine. The composition may also contain a local anesthetic with or without epinephrine. The composition is delivered using a dental syringe typically used for administering local anesthesia. The needle is inserted below the submucosally and the mixture is delivered via local infiltration adjacent the procedure site at the preoperative or intraoperative stage. The local infiltration of the medication provides improved postoperative pain control, reducing swelling and decreasing the need for postoperative opioid consumption. Ketorolac tromethamine may also be administered after the procedure.

### CROSS REFERENCE TO RELATED APPLICATION

This application claims the benefit of U.S. Provisional Application No. 63/559,542 filed Feb. 29, 2024, which is hereby incorporated herein by reference in its entirety.

### BACKGROUND OF THE DISCLOSURE

The present disclosure relates to medical compositions, more specifically to medicinal compositions for use in oral surgery procedures.

Many invasive oral procedures, for example third molar extractions, require the use of drugs to help with postoperative swelling and pain. Often, opioids, such as Vicodin, are prescribed to manage postoperative pain. Although beneficial for reducing pain, opioids are highly addictive. Individuals may become addicted to the opioids they are legally prescribed, leading to an opioid crisis in the United States and around the world. In 2017, more than 191 million opioid prescriptions were dispensed in the United States alone. In 2016, more than 11.5 million Americans reported misusing prescription opioids within the last year. According to the Centers for Disease Control and Prevention statistics, over 75% of the nearly 107,000 drug overdose deaths in 2021 involved an opioid. Besides death, there are numerous negative physical and social side effects of opioid addiction. Even if a person does not become addicted to opioids, opioid consumption has many adverse side effects including nausea, vomiting, constipation, itching, etc. Non-opioid medical compositions to help replace opioids and decrease the need for opioid consumption are desirable.

Thus, there is a need for improvement in this field.

### SUMMARY OF THE DISCLOSURE

One or more unique non-opioid formulations/compositions have been developed to treat patient pain while decreasing the need for opioid consumption. Moreover, one or more processes for preparing/manufacturing and administering the formulations are disclosed. The Applicant has discovered that preoperative or intraoperative, local infiltration of the disclosed composition provides better postoperative pain control and thus, decreases the need for postoperative narcotic consumption. It is understood that the disclosed compositions may be administered for pain control purposes for a number of invasive medical procedures. For representative purposes only, the composition is discussed in relation to the field of dentistry.

In one example, a composition may include dexamethasone, and ketorolac tromethamine, often simply referred to as ketorolac. Dexamethasone is a glucocorticoid steroid which reduces a patient's swelling/inflammation. Ketorolac tromethamine is a nonsteroidal anti-inflammatory drug (NSAID) used to treat pain, and is typically administered via an intravenous (IV) drip or an intramuscular (IM) injection.

Thus, in accordance with some forms, the present disclosure provides a non-opioid anesthetic composition comprising dexamethasone and ketorolac tromethamine. As discussed herein, such composition may be provided as separate components to be mixed prior to administration. Alternatively, the composition may be provided as a preformulated composition. For example, in certain embodiments the present disclosure provides for a sealed container, such as a carpule, single-dose vial or multi-dose vial, containing a non-opioid anesthetic composition as provided herein.

In use, the compositions disclosed herein may be administered to a patient, for example a patient undergoing a dental procedure such as oral surgery. Such administration may comprise injection of the composition into the submucosal tissue at or near the site of the procedure, for example using a syringe. In certain embodiments, administration comprises a subdermal and/or intramuscular injection of the composition.

In certain embodiments, the present disclosure provides for a composition including a solution with at least about a 1-20 mg/mL concentration of dexamethasone, more typically about a 4-15 mg/mL concentration of dexamethasone and even more preferred about an 8-12 mg/mL concentration of dexamethasone.

The composition may also include a solution with at least about a 1-40 mg/mL concentration of ketorolac tromethamine, more typically about a 10-35 mg/mL concentration of ketorolac tromethamine and even more preferred about a 25-35 mg/mL concentration of ketorolac tromethamine.

In some exemplary embodiments, a solution with a concentration of about 10 mg/mL of dexamethasone and a solution with a concentration of about 30 mg/mL of ketorolac tromethamine may be mixed to create the composition. These ingredients may be mixed using any known method of mixing, including but not limited to swirling or stirring. In other embodiments, the ingredients are loaded into a syringe or cartridge without any active physical mixing.

In some embodiments, the composition may include a 1:1 ratio of a solution with a concentration of 10 mg/mL of dexamethasone and a solution with a concentration of about 30 mg/mL of ketorolac tromethamine, respectively. In some exemplary embodiments, the composition may include about 1 mL of a solution with a concentration of about 10 mg/mL of dexamethasone and about 1 mL of a solution with a concentration of about 30 mg/mL of ketorolac tromethamine.

1 mL: 1 mL ratios of the various solution concentrations disclosed above may also be used to create the composition. In other embodiments, other therapeutic ratios may be used, for example 1:2 or 2:1 of a dexamethasone solution according to the concentrations discussed above to ketorolac tromethamine solution according to the concentrations discussed above.

In exemplary embodiments, the composition may include about 10 mg of dexamethasone to about 30 mg of ketorolac tromethamine or a 1:3 ratio of dexamethasone to ketorolac tromethamine by weight. Other ratios of dexamethasone to ketorolac that may be provided including 1:1, 1:2, 1:4, and 1:5. Based on the solution concentrations discussed above, and depending on the volume of each solution utilized, other weight ratios of dexamethasone to ketorolac tromethamine may be used.

In some examples, the composition is composed of about 0.05%-1% dexamethasone by total volume, more typically the composition is composed of about 0.2%-0.75% dexamethasone by total volume and even more preferred the composition is composed of about 0.4%-0.6% dexamethasone by total volume. In some exemplary embodiments the composition is composed of about 0.5% dexamethasone by total volume.

In some examples, the composition is composed of about 0.05%-2% ketorolac tromethamine by total volume, more typically the composition is composed of about 0.5%-1.75% ketorolac tromethamine by total volume and even more preferred the composition is composed of about 1.25%-1.75% ketorolac tromethamine by total volume. In some exemplary embodiments the composition is composed of about 1.5% ketorolac tromethamine by total volume.

The composition may include about 1-20 mg of dexamethasone, more typically about 4-15 mg of dexamethasone and even more preferred about 8-12 mg of dexamethasone.

The composition may also include about 1-40 mg of ketorolac tromethamine, more typically about 10-35 mg of ketorolac tromethamine and even more preferred about 25-35 mg of ketorolac tromethamine.

In accordance with some forms, the present disclosure provides methods of administering a therapeutic dose dexamethasone and ketorolac tromethamine. In certain embodiments, a therapeutic dose comprises about 1-20 mg of dexamethasone, preferably about 4-15 mg of dexamethasone and even more preferred about 8-12 mg of dexamethasone. In certain embodiments, a therapeutic dose comprises about 1-40 mg of ketorolac tromethamine, more preferably about 10-35 mg of ketorolac tromethamine and even more preferred about 25-35 mg of ketorolac tromethamine.

In some embodiments, the composition comprises inactive ingredients. In some forms the dexamethasone solution may include sodium sulfite anhydrous, sodium citrate anhydrous and benzyl alcohol in water. The pH of the composition may be adjusted, for example with citric acid, sodium hydroxide and/or hydrochloric acid. Other ingredients may include polyethylene glycol 400, preservatives (e.g. methylparaben and propylparaben), and alcohol.

In other embodiments, each mL of dexamethasone solution may contain dexamethasone sodium phosphate, sodium citrate, dihydrate; and water for injection. The pH may be adjusted by adding an additional base or acid, for example citric acid or sodium hydroxide.

The excipients in the ketorolac solution may include ethanol, sodium chloride and water for injections. The desired pH range is about 6.9 to 7.9 and is adjusted with a base or acid, for example sodium hydroxide and/or hydrochloric acid.

In some embodiments, the composition may further include a local anesthetic. In some exemplary embodiments, the local anesthetic may be lidocaine. In other embodiments, the local anesthetic may be mepivacaine, bupivacaine, prilocaine, or articaine. Commercially available formulations of lidocaine may be used including Lidocaine 2% and epinephrine 1:50,000 or lidocaine 2% 1:100,00 with epinephrine. Although typically used with epinephrine, lidocaine without epinephrine may be used. Typically, for normal healthy adults, the amount of lidocaine included in the composition is below about 500 mg or more typically about 7 mg/kg (3.4 mg/lb). However, amounts below therapeutic value levels may be used.

In some examples, the composition may further include a flavoring agent.

The composition may be contained within a sterile package/kit. The kit may contain any number of the components discussed above. In one example, the kit may include a sterilely sealed vial of dexamethasone and a separate sterilely sealed vial of ketorolac tromethamine. The sterilely sealed vials may contain at least about 0.1 mL of dexamethasone at a concentration of about 10 mg/mL and at least about 0.1 mL of ketorolac tromethamine at a concentration of about 30 mg/mL, respectively. In another example, the kit may include a sterilely sealed vial of dexamethasone, a separate sterilely sealed vial of ketorolac tromethamine, and a separate sterilely sealed vial of lidocaine with or without epinephrine. In yet another embodiment, the kit may include a sterilely sealed vial of dexamethasone, a separate sterilely sealed vial of ketorolac tromethamine, a separate sterilely sealed vial of lidocaine with or without epinephrine, and a sterile syringe, for example a dental syringe. It is noted that any number of each component may come in each kit. For example, a single sterilely sealed vial of dexamethasone and a single sterilely sealed vial of ketorolac tromethamine may come in each kit. In other embodiments, 2 or more sterilely sealed vials of dexamethasone and sterilely sealed vials of ketorolac tromethamine may be included in each kit.

In another example, a kit may include a sterilely sealed cartridge, such as a carpule, single-use vial or multi-use vial, containing a composition including dexamethasone and ketorolac tromethamine. The composition may be formulated according to any embodiment described above. The cartridge may further include a local anesthetic, such as lidocaine with or without epinephrine. In other embodiments, a separate container/vial of lidocaine may be included in the kit. The kit may further include a syringe, for example a dental syringe. It is noted that any number of each component may come in each kit. For example, a single sterilely sealed cartridge of the composition may come in each kit. In other embodiments, 2 or more cartridges may be included in each kit.

Further disclosed is a method for preparing and administering the composition to a patient. It is understood that the stages as sequenced are not meant to be limiting and may be performed in any reasonable order. First, a solution containing dexamethasone is added to a syringe or container. Then, a solution containing ketorolac tromethamine is added to the same container for mixing. In some examples, about 1 cc of a solution with a concentration of about 10 mg/mL dexamethasone and about 1 cc of a solution with a concentration of about 30 mg/mL ketorolac tromethamine are added to the container.

In some embodiments, lidocaine with or without epinephrine may also be added to the composition.

In other embodiments, a flavoring agent may be added to the composition.

There are numerous methods to prepare the composition. In one example, an individual may use a sterilely filled vial of a solution containing dexamethasone and a sterilely filled vial of a solution containing ketorolac tromethamine. The user may load the composition into the syringe by extracting the solution from the vial containing dexamethasone and by extracting the solution from the vial containing ketorolac tromethamine into the same syringe. In some embodiments, a solution containing lidocaine may also be loaded into the same syringe. In some examples, one or more of the ingredients may be mixed prior to loading into the syringe. In another example, the user may inject the contents of the syringe into a cartridge or carpule to then be administered using a traditional dental syringe or a dental aspirating syringe compatible with a cartridge/carpule.

In yet another example, when manufactured, the ingredients may be added to a sterilely filled and sealed cartridge for storage or transportation purposed. The sterilely filled cartridge may be designed for use with any dental syringe known in the art. The cartridge may be a carpule, single-dose vial or multi-dose vial. The composition may be mixed prior to loading it into a cartridge. In other examples, each individual ingredient may be added to the cartridge individually to create the composition.

Next, the composition as formulated above is administered to a patient prior to a surgical procedure. In one example, the administrator of the composition may locally deliver the composition to the patient directly from the syringe the composition was mixed in. In other examples, the composition may be administered to the patient from a cartridge containing the composition using a dental syringe.

In some exemplary embodiments, the composition is administered prior to an invasive dental procedure. The composition may be delivered submucosally via local infiltration adjacent to the procedure site. Infiltration adjacent to the site of the procedure may reduce postoperative swelling and pain. In some examples, a topical anesthetic may be used prior to the delivery of the composition. In other examples, a local anesthetic may be delivered adjacent the site of the procedure prior to the administering the composition.

Finally, to minimize the amount of postoperative pain and inflammation, a patient may be administered 2 doses/pills of ketorolac tromethamine following the procedure. The patient may be administered a single dosage every 6 hours for 5 days following the administration of the original 2 doses.

Further forms, objects, features, aspects, benefits, advantages, and embodiments of the present disclosure will become apparent from a detailed description and drawings provided herewith.

### BRIEF DESCRIPTION OF THE DRAWINGS

FIG. 1 is a schematic diagram of a composition according to the present disclosure sterilely packaged in a kit.

FIG. 2 is a flowchart of a method of preparing a non-opioid composition according to the present disclosure and administering the composition to a patient.

### DETAILED DESCRIPTION OF THE DISCLOSURE

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which the disclosure relates. One embodiment of the disclosure is shown in great detail, although it will be apparent to those skilled in the relevant art that some features that are not relevant to the present disclosure may not be shown for the sake of clarity.

After an invasive dental procedure, patients are often prescribed opioids or steroids in order to deal with swelling and/or pain. Unfortunately, opioids are highly addictive and addiction can lead to numerous negative social and physical side effects, including an overdose death. Other side effects include nausea, vomiting, constipation, itching, among others. In order to decrease the need for opioid use and therefore addiction, alternative treatments and drugs are necessary to replace traditional opioids.

One or more unique non-opioid formulations/compositions have been developed to treat patient pain while decreasing the need for opioid consumption. Moreover, one or more processes for preparing/manufacturing and administering the formulations are disclosed. The Applicant has discovered that preoperative or intraoperative, local infiltration of the disclosed composition provides better postoperative pain control and thus, decreases the need for postoperative narcotic consumption. The synergistic effect of the composition of drugs, as well as the local infiltration of the composition, provides better postoperative pain control than prior methods of administration and/or administering the components of the composition separately. It is understood that the disclosed compositions may be administered for pain control purposes for a number of invasive medical procedures. For representative purposes only, the composition is discussed in relation to the field of dentistry.

In one example, a composition may include dexamethasone, and ketorolac tromethamine, often simply referred to as ketorolac. Dexamethasone is a glucocorticoid steroid which reduces a patient's swelling/inflammation. Ketorolac tromethamine is a nonsteroidal anti-inflammatory drug (NSAID) used to treat pain. Ketorolac is typically administered via an intravenous (IV) drip or an intramuscular (IM) shot.

Thus, in accordance with some forms, the present disclosure provides a non-opioid anesthetic composition comprising dexamethasone and ketorolac tromethamine. As discussed herein, such composition may be provided as separate components to be mixed prior to administration. Alternatively, the composition may be provided as a preformulated composition. For example, in certain embodiments the present disclosure provides for a sealed container, such as a carpule, single-dose vial or multi-dose vial, containing a non-opioid anesthetic composition as provided herein. The multi-dose vial may be any reasonable size.

In use, the compositions disclosed herein may be administered to a patient, for example a patient undergoing a dental procedure such as oral surgery. Such administration may comprise injection of the composition into the submucosal tissue at or near the site of the procedure, for example using a syringe. In this way, the methods of the present disclosure include methods of performing oral surgery, or methods of anesthetizing a patient prior to an oral surgical procedure, the methods comprising the step of administering a therapeutic dose of any of the compositions disclosed herein. However, in other embodiments, the injection may comprise a subcutaneous and/or intramuscular injection.

In certain embodiments, the present disclosure provides for a composition including a dexamethasone solution with at least about a 1-20 mg/mL concentration of dexamethasone, more typically about a 4-15 mg/mL concentration of dexamethasone and even more preferred about an 8-12 mg/mL concentration of dexamethasone.

The composition may also include a ketorolac tromethamine solution with at least about a 1-40 mg/mL concentration of ketorolac tromethamine, more typically about a 10-35 mg/mL concentration of ketorolac tromethamine and even more preferred about a 25-35 mg/mL concentration of ketorolac tromethamine.

In some exemplary embodiments, a dexamethasone solution with a concentration of about 1 mg/mL to about 20 mg/mL of dexamethasone and a ketorolac tromethamine solution with a concentration of about 1 mg/mL to about 40 mg/mL of ketorolac tromethamine may be mixed to create the composition. In one embodiment, a dexamethasone solution with a concentration of about 10 mg/mL of dexamethasone and a ketorolac tromethamine solution with a concentration of about 30 mg/mL of ketorolac tromethamine may be mixed to create the composition. These ingredients may be mixed using any known method of mixing, including but not limited to swirling or stirring. In other embodiments, the ingredients are loaded into a syringe or cartridge without any active physical mixing.

In some embodiments, the composition may include a 1:1 ratio by volume of a solution with a concentration of 10 mg/mL of dexamethasone and a solution with a concentration of about 30 mg/mL of ketorolac tromethamine, respectively. In some exemplary embodiments, the composition may include 1 mL of a solution with a concentration of about 10 mg/mL of dexamethasone and 1 mL of a solution with a concentration of about 30 mg/mL of ketorolac tromethamine.

1 mL: 1 mL ratios of the various solution concentrations disclosed above may also be used to create the composition. In other embodiments, other therapeutic ratios may be used, for example 1:2 or 2:1 of a dexamethasone solution according to the concentrations discussed above to ketorolac tromethamine solution according to the concentrations discussed above.

In exemplary embodiments, the composition may include 10 mg of dexamethasone to about 30 mg of ketorolac tromethamine or a 1:3 ratio of dexamethasone to ketorolac tromethamine by weight. Other weight ratios of dexamethasone to ketorolac that may be provided include 1:1, 1:2, 1:4, and 1:5. Based on the solution concentrations discussed above, and depending on the volume of each solution utilized, other weight ratios of dexamethasone to ketorolac tromethamine may be used.

In some examples, the composition is composed of about 0.05%-1% dexamethasone by total volume, more typically the composition is composed of about 0.2%-0.75% dexamethasone by total volume and even more preferred the composition is composed of about 0.4%-0.6% dexamethasone by total volume. In some exemplary embodiments the composition is composed of about 0.5% dexamethasone by total volume.

In some examples, the composition is composed of about 0.05%-2% ketorolac tromethamine by total volume, more typically the composition is composed of about 0.5%-1.75% ketorolac tromethamine by total volume and even more preferred the composition is composed of about 1.25%-1.75% ketorolac tromethamine by total volume. In some exemplary embodiments the composition is composed of about 1.5% ketorolac tromethamine by total volume.

The composition may include about 1-20 mg of dexamethasone, more typically about 4-15 mg of dexamethasone and even more preferred about 8-12 mg of dexamethasone.

The composition may also include about 1-40 mg of ketorolac tromethamine, more typically about 10-35 mg of ketorolac tromethamine and even more preferred about 25-35 mg of ketorolac tromethamine.

In accordance with some forms, the present disclosure provides methods of administering a therapeutic dose of a composition comprising dexamethasone and ketorolac tromethamine. In certain embodiments, a therapeutic dose comprises about 1-20 mg of dexamethasone, preferably about 4-15 mg of dexamethasone and even more preferred about 8-12 mg of dexamethasone. In certain embodiments, a therapeutic dose comprises about 1-40 mg of ketorolac tromethamine, more preferably about 10-35 mg of ketorolac tromethamine and even more preferred about 25-35 mg of ketorolac tromethamine.

In some embodiments, the composition comprises inactive ingredients. In some forms the dexamethasone solution may include sodium sulfite anhydrous, sodium citrate anhydrous and benzyl alcohol in water. The pH of the composition may be adjusted, for example with citric acid, sodium hydroxide and/or hydrochloric acid. Other ingredients may include polyethylene glycol 400, preservatives (e.g. methylparaben and propylparaben), and alcohol.

In other embodiments, each mL of dexamethasone solution may contain dexamethasone sodium phosphate, sodium citrate, dihydrate; and water for injection. The pH may be adjusted by adding an additional base or acid, for example citric acid or sodium hydroxide.

The excipients in the ketorolac solution may include ethanol, sodium chloride and water for injections. The desired pH range is about 6.9 to 7.9 and is adjusted with a base or acid, for example sodium hydroxide and/or hydrochloric acid.

In some embodiments, the composition may further include a local anesthetic. In some exemplary embodiments, the local anesthetic may be lidocaine. In other embodiments, the local anesthetic may be mepivacaine, bupivacaine, prilocaine, or articaine. Commercially available formulations of lidocaine may be used including Lidocaine 2% and epinephrine 1:50,000 or lidocaine 2% 1:100,00 with epinephrine. Although typically used with epinephrine, lidocaine without epinephrine may be used. Typically, for normal healthy adults, the amount of lidocaine included in the composition is below about 500 mg or more typically about 7 mg/kg (3.4 mg/lb). However, amounts below therapeutic value levels may be used.

In some examples, the composition may further include a flavoring agent.

The following specific Examples are provided to promote a further understanding of certain aspects of the present disclosure. It will be understood that these Examples are illustrative, and not limiting, in character.

### Example 1

A composition including a solution with a concentration of about 10 mg/mL of dexamethasone and a solution with a concentration of about 30 mg/mL of ketorolac tromethamine was prepared. The solutions were mixed/loaded in a cartridge/syringe. The composition was pH adjusted to the desired pH range is about 6.9 to 7.9 with sodium hydroxide and/or hydrochloric acid. The composition was loaded into a cartridge compatible with a dental syringe and then submucosally delivered to a patent undergoing an oral surgical procedure adjacent the site of the procedure.

### Example 2

A composition including a solution with a concentration of about 10 mg/mL of dexamethasone, a solution with a concentration of about 30 mg/mL of ketorolac tromethamine and a solution with a concentration of up to about 500 mg of lidocaine was prepared. These solutions were mixed/loaded in a container/syringe. The composition was administered to a patient undergoing an oral surgical procedure with a dental syringe into the submucosal tissue adjacent the site of the procedure.

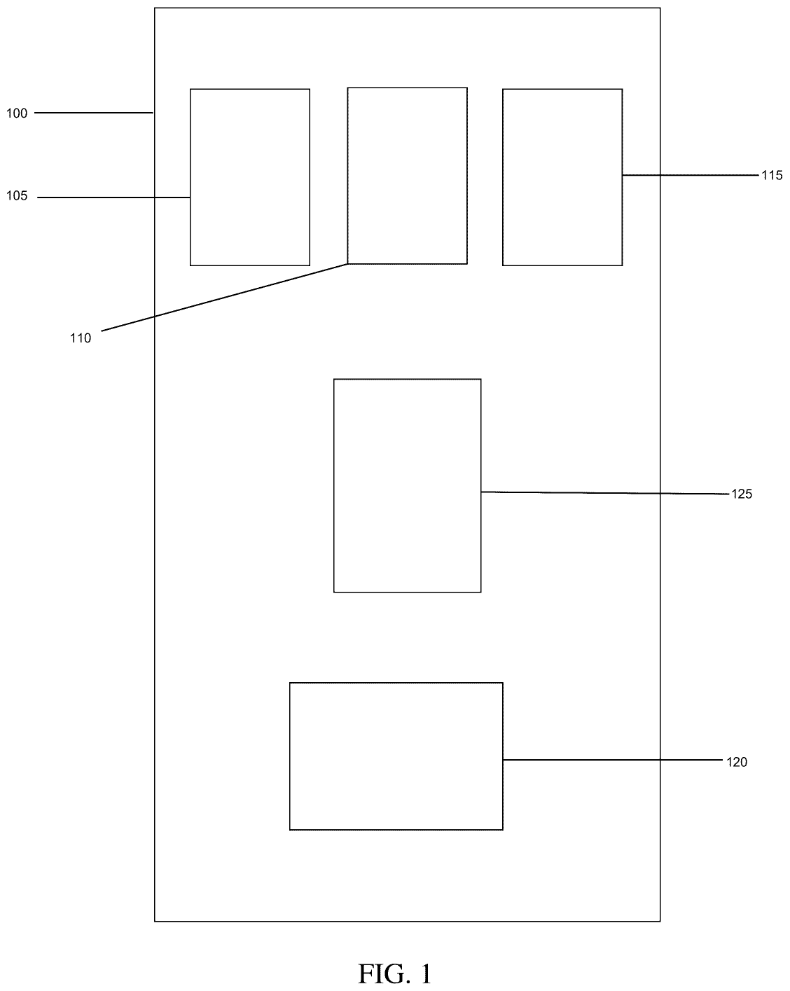

Turning to FIG. 1, the composition may be contained within a sterile package/kit 100. The kit 100 may contain any number of the components discussed above. In one example, the kit 100 may include a sterilely sealed vial/container of dexamethasone 105 and a separate sterilely sealed vial/container of ketorolac tromethamine 110. The sterilely sealed vials 105, 110 may contain at least about 0.1 mL of dexamethasone at a concentration of about 10 mg/mL and at least about 0.1 mL of ketorolac tromethamine at a concentration of about 30 mg/mL, respectively. In another example, the kit 100 may include a sterilely sealed vial of dexamethasone 105, a separate sterilely sealed vial of ketorolac tromethamine 110, and a separate sterilely sealed vial of lidocaine 115 with or without epinephrine. In yet another embodiment, the kit 100 may include a sterilely sealed vial of dexamethasone 105, a separate sterilely sealed vial of ketorolac tromethamine 110, a separate sterilely sealed vial of lidocaine 115 with or without epinephrine, and a sterile syringe 120, for example a dental syringe. It is noted that any number of each component may come in each kit 100. For example, a single sterilely sealed vial of dexamethasone 105 and a single sterilely sealed vial of ketorolac tromethamine 110 may come in each kit 100. In other embodiments, 2 or more sterilely sealed vials of dexamethasone 105 and sterilely sealed vials of ketorolac tromethamine 110 may be included in each kit 100.

In another example, a kit 100 may include a sterilely sealed cartridge 125, such as a carpule, single-dose vial or multi-dose vial, containing a composition including dexamethasone and ketorolac tromethamine. The composition may be formulated according to any embodiment described above. The cartridge 125 may further include a local anesthetic, such as lidocaine with or without epinephrine. In other embodiments, a separate container/vial of lidocaine 115 may be included in the kit 100. The kit 100 may further include a syringe 120, for example a dental syringe. It is noted that any number of each component may come in each kit 100. For example, a single sterilely sealed cartridge 125 of the composition may come in each kit 100. In other embodiments, 2 or more cartridges 125 may be included in each kit 100.

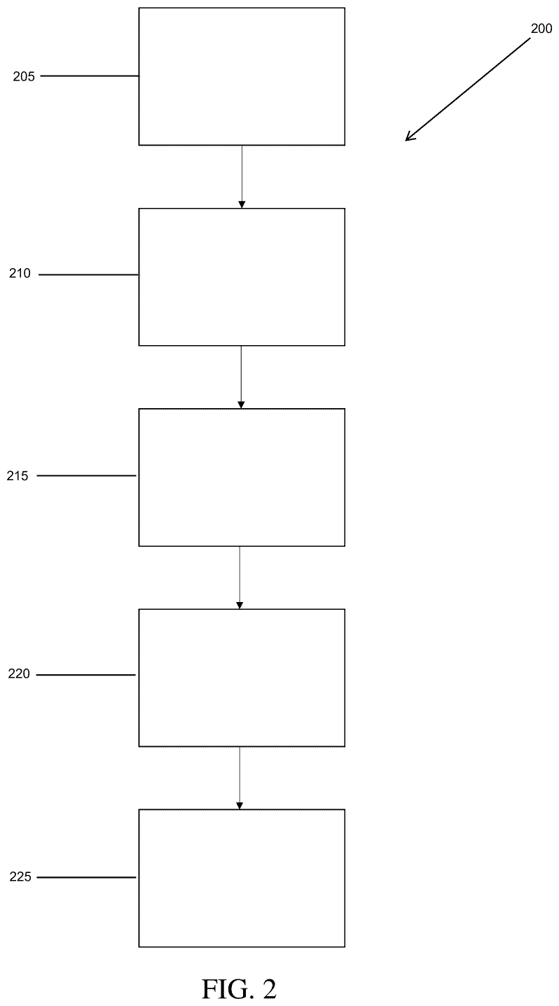

FIG. 2 illustrates flowchart 200 of a method for preparing and administering the composition to a patient. It is understood that the stages as sequenced are not meant to be limiting and may be performed in any reasonable order. In stage 205, a solution containing dexamethasone is added to a syringe or container. In stage 210, a solution containing ketorolac tromethamine is added to the same container for mixing. In some examples, about 1 cc of a solution with a concentration of about 10 mg/mL dexamethasone and about 1 cc of a solution with a concentration of about 30 mg/mL ketorolac tromethamine are added to the container.

In some embodiments, in stage 215, lidocaine with or without epinephrine may also be added to the composition.

There are numerous methods to accomplish stages 205, 210 and 215. In one example, an individual may use a sterilely filled vial of a solution containing dexamethasone and a sterilely filled vial of a solution containing ketorolac tromethamine. The user may load the composition into the syringe by extracting the solution from the vial containing dexamethasone and by extracting the solution from the vial containing ketorolac tromethamine into the same syringe. In some embodiments, a solution containing lidocaine may also be loaded into the same syringe. In some examples, one or more of the ingredients may be mixed prior to loading into the syringe. In another example, the user may inject the contents of the syringe into a cartridge or carpule to then be administered using a traditional dental syringe or a dental aspirating syringe compatible with a cartridge/carpule.

In yet another example, when manufactured, the ingredients may be added to a sterilely filled and sealed cartridge for storage or transportation purposed. The sterilely filled cartridge may be designed for use with any dental syringe known in the art. The cartridge may be a carpule, single-dose vial or multi-dose vial. The composition may be mixed prior to loading it into a cartridge. In other examples, each individual ingredient may be added to the cartridge individually to create the composition.

In stage 220, the composition as formulated above is administered to a patient prior to a surgical procedure. In one example, the administrator of the composition may locally deliver the composition to the patient directly from the syringe the composition was mixed in. In other examples, the composition may be administered to the patient from a cartridge containing the composition using a dental syringe. In other embodiments, the composition may be administered to the patient after extracting the composition from a single-dose vial or multi-dose vial into an injection device, such as a syringe.

In some exemplary embodiments, the composition is administered prior to an invasive dental procedure. The composition may be delivered submucosally/subperiosteal via local infiltration adjacent to the procedure site. Infiltration adjacent to the site of the procedure may reduce postoperative swelling and pain. In some examples, a topical anesthetic may be used prior to the delivery of the composition. In other examples, a local anesthetic may be delivered adjacent the site of the procedure prior to the administering the composition.

In some examples, administration of the disclosed compositions prior to an invasive dental procedure or at the preoperative stage may mean about 1 second to about 60 minutes prior to the procedure, more typically about 1 minute to about 30 minutes prior to the procedure and even more preferred less than about 5 minutes prior to the procedure.

In accordance with some embodiments, a first dose or a subsequent dose of the disclosed compositions may be administered after the procedure has begun or after the first cutting of tissue. In yet other examples, the disclosed compositions may be administered at the postoperative stage or after the procedure has concluded.

In stage 225, to minimize the amount of postoperative pain and inflammation, a patient may be administered 2 doses/pills of ketorolac tromethamine following the procedure. The patient may be administered a single dosage every 6 hours for 5 days following the administration of the original 2 doses.

As used herein, mixing may mean some form of active physical mixing of the ingredients in the composition. In some embodiments, mixing means the natural mixing of the ingredients that occurs simply by being contained within the same container.

As used herein, container may mean a syringe, a cartridge, a carpule, an ampule, Erlenmeyer flasks, a beaker, round-bottom flasks, a mixing tube or any other storage container capable of holding liquid for mixing purposes. The mixing can happen in any of these containers, including the end cartridge for storage and transportation. A carpule is a small cylindrical glass tube connectable to a dental syringe. Often, the carpule is connected to the dental syringe by screwing the carpule onto a dental syringe. Commonly, carpules are aspirated while the practitioner injects a patient. A carpule may be a 1.7 mL glass carpule. Although most carpules are currently 1.7 mL, it is understood that the disclosed compositions may be utilized at any appropriate volume.

“About” with reference to numerical values generally refers to plus or minus 10% of the stated value. For example, if the stated value is 4.375, then use of the term “about 4.375” generally means a range between 3.9375 and 4.8125.

It is noted stat all stated values may be exactly or essentially the stated values.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the disclosures defined by following claims are desired to be protected. All publications, patents, and patent applications cited in this specification are herein incorporated by reference as if each individual publication, patent, or patent application were specifically and individually indicated to be incorporated by reference and set forth in its entirety herein.

### Clauses

The following numbered clauses set out specific embodiments that may be useful in understanding the present disclosure:

Clause 1: A composition, comprising: dexamethasone; and ketorolac tromethamine.

Clause 2: The composition of clause 1, further comprising: a local anesthetic.

Clause 3: The composition of any preceding clause, wherein the local anesthetic is lidocaine.

Clause 4: The composition of any preceding clause, further comprising: a flavoring agent.

Clause 5: The composition of any preceding clause, wherein the composition includes about 10 mg of dexamethasone.

Clause 6: The composition of any preceding clause, wherein the composition includes about 30 mg of ketorolac tromethamine.

Clause 7: The composition of any preceding clause, wherein the composition is administered beneath the oral mucosa prior to invasive oral surgery.

Clause 8: A kit sterilely received in a packaging, comprising: a sterilely filled cartridge for use with a dental syringe, containing: dexamethasone; and ketorolac tromethamine.

Clause 9: The kit of clause 7, wherein the sterilely filled cartridge further contains a local anesthetic.

Clause 10: The kit of any preceding clause, wherein the local anesthetic is lidocaine.

Clause 11: The kit of any preceding clause, wherein the sterilely filled cartridge further contains a flavoring agent.

Clause 12: The kit of any preceding clause, wherein the composition includes about 10 mg of dexamethasone.

Clause 13: The kit of any preceding clause, wherein the composition includes about 30 mg of ketorolac tromethamine.

Clause 14: The kit of any preceding clause, further comprising: a sterile dental syringe.

Clause 15: A method of preparing an injectable composition, comprising: adding dexamethasone to a container; adding ketorolac tromethamine to the container.

Clause 16: The method of clause 13, further comprising: adding a local anesthetic to the container.

Clause 17: The method of any preceding clause, wherein the local anesthetic is lidocaine.

Clause 18: The method of any preceding clause, further comprising: adding a flavoring agent to the container.

Clause 19: The method of any preceding clause, wherein the composition includes about 10 mg of dexamethasone.

Clause 20: The method of any preceding clause, wherein the composition includes about 30 mg of ketorolac tromethamine.

Clause 21: The method of any preceding clause, further comprising: transferring the composition into a cartridge.

Clause 22: The method of any preceding clause, wherein the container is a syringe.

Clause 23: The method of any preceding clause, wherein the container is a cartridge.

Clause 24: The method of any proceeding clause, wherein the composition in administered submucosally or subperiosteal via local infiltration.

Clause 25: The method of any proceeding clause, wherein the composition in administered via a dental aspirating syringe.

Clause 26: A method of preparing and administering a composition, comprising: adding about 10 mg of dexamethasone to a cartridge to mix into a composition; adding about 30 mg ketorolac tromethamine to the cartridge to mix into the composition; adding a local anesthetic to the cartridge to mix into the composition; connecting the cartridge to a dental syringe; and administering the composition submucosally or subperiosteal via local infiltration.

Clause 27: The composition/kit/method of any preceding clause, wherein the dexamethasone is a solution with at least about a 1-20 mg/mL concentration of dexamethasone, more typically about a 4-15 mg/mL concentration of dexamethasone and even more preferred about an 8-12 mg/mL concentration of dexamethasone.

Clause 28: The composition/kit/method of any preceding clause, wherein the ketorolac tromethamine is a solution with at least about a 1-40 mg/mL concentration of ketorolac tromethamine, more typically about a 10-35 mg/mL concentration of ketorolac tromethamine and even more preferred about a 25-35 mg/mL concentration of ketorolac tromethamine.

Clause 29: The composition/kit/method of any preceding clause, wherein the dexamethasone is a solution with a concentration of about 10 mg/mL of dexamethasone and the ketorolac tromethamine is a solution with a concentration of about 30 mg/mL of ketorolac tromethamine.

Clause 30: The composition/kit/method of any preceding clause, wherein the composition includes a 1:1 ratio of a solution with a concentration of about 10 mg/mL of dexamethasone and a solution with a concentration of about 30 mg/mL of ketorolac tromethamine.

Clause 31: The composition/kit/method of any preceding clause, wherein the composition may include about 1 mL of a solution with a concentration of about 10 mg/mL of dexamethasone and about 1 mL of a solution with a concentration of about 30 mg/mL of ketorolac tromethamine.

Clause 32: The composition/kit/method of any preceding clause, wherein the composition includes about 10 mg of dexamethasone to about 30 mg of ketorolac tromethamine.

Clause 33: The composition/kit/method of any preceding clause, wherein the composition is composed of about 0.05%-1% dexamethasone by total volume, more typically the composition is composed of about 0.2%-0.75% dexamethasone by total volume and even more preferred the composition is composed of about 0.4%-0.6% dexamethasone by total volume.

Clause 34: The composition/kit/method of any preceding clause, wherein the composition is composed of about 0.5% dexamethasone by total volume.

Clause 35: The composition/kit/method of any preceding clause, wherein the composition is composed of about 0.05%-2% ketorolac tromethamine by total volume, more typically the composition is composed of about 0.5%-1.75% ketorolac tromethamine by total volume and even more preferred the composition is composed of about 1.25%-1.75% ketorolac tromethamine by total volume.

Clause 36: The composition/kit/method of any preceding clause, wherein the composition is composed of about 1.5% ketorolac tromethamine by total volume.

Clause 37: The composition/kit/method of any preceding clause, wherein the composition may include about 1-20 mg of dexamethasone, more typically about 4-15 mg of dexamethasone and even more preferred about 8-12 mg of dexamethasone.

Clause 38: The composition/kit/method of any preceding clause, the composition may also include about 1-40 mg of ketorolac tromethamine, more typically about 10-35 mg of ketorolac tromethamine and even more preferred about 25-35 mg of ketorolac tromethamine.

Clause 39: A cartridge connectable to a dental syringe containing a non-opioid composition, comprising: dexamethasone; ketorolac tromethamine; a local anesthetic.

Clause 40: The cartridge connectable to a dental syringe containing a non-opioid composition of clause 39, wherein the local anesthetic is lidocaine.

Clause 41: cartridge connectable to a dental syringe containing a non-opioid composition of any preceding clause, further comprising a flavoring agent.

Clause 42: The cartridge connectable to a dental syringe containing a non-opioid composition of any preceding clause, wherein the composition includes about 10 mg of dexamethasone.

Clause 43: The cartridge connectable to a dental syringe containing a non-opioid composition of any preceding clause, wherein the composition includes about 30 mg of ketorolac tromethamine.

Clause 44: The cartridge connectable to a dental syringe containing a non-opioid composition of any preceding clause, wherein the composition is administered beneath the oral mucosa via local infiltration prior to invasive oral surgery.

Clause 45: A method of reducing postoperative pain after a dental procedure, comprising: adding dexamethasone to a cartridge to mix into a composition; adding ketorolac tromethamine to the cartridge to mix into the composition; adding a local anesthetic to the cartridge to mix into the composition; connecting the cartridge to a dental syringe; and administering the composition submucosally or subperiosteal via local infiltration.

Clause 46: The method of reducing postoperative pain after a dental procedure of any preceding clause, wherein the composition is administered about 1 second to 60 minutes prior to the procedure, more typically about 1 minute to 30 minutes prior to the procedure and even more preferred less than about 5 minutes prior to the procedure.

Clause 47: The method of reducing postoperative pain after a dental procedure of any preceding clause, wherein the dexamethasone is a solution with at least about a 1-20 mg/mL concentration of dexamethasone, more typically about a 4-15 mg/mL concentration of dexamethasone and even more preferred about an 8-12 mg/mL concentration of dexamethasone.

Clause 48: The method of reducing postoperative pain after a dental procedure of any preceding clause, wherein the ketorolac tromethamine is a solution with at least about a 1-40 mg/mL concentration of ketorolac tromethamine, more typically about a 10-35 mg/mL concentration of ketorolac tromethamine and even more preferred about a 25-35 mg/mL concentration of ketorolac tromethamine.

Clause 49: The method of reducing postoperative pain after a dental procedure of any preceding clause, wherein the dexamethasone is a solution with a concentration of about 10 mg/mL of dexamethasone and the ketorolac tromethamine is a solution with a concentration of about 30 mg/mL of ketorolac tromethamine.

Clause 50: The method of reducing postoperative pain after a dental procedure of any preceding clause, wherein the composition includes a 1:1 ratio of a solution with a concentration of about 10 mg/mL of dexamethasone and a solution with a concentration of about 30 mg/mL of ketorolac tromethamine.

Clause 51: The method of reducing postoperative pain after a dental procedure of any preceding clause, wherein the composition may include about 1 mL of a solution with a concentration of about 10 mg/mL of dexamethasone and about 1 mL of a solution with a concentration of about 30 mg/mL of ketorolac tromethamine.

Clause 52: The method of reducing postoperative pain after a dental procedure of any preceding clause, wherein the composition includes about 10 mg of dexamethasone to about 30 mg of ketorolac tromethamine.

Clause 53: The method of reducing postoperative pain after a dental procedure of any preceding clause, wherein the composition is composed of about 0.05%-1% dexamethasone by total volume, more typically the composition is composed of about 0.2%-0.75% dexamethasone by total volume and even more preferred the composition is composed of about 0.4%-0.6% dexamethasone by total volume.

Clause 54: The method of reducing postoperative pain after a dental procedure of any preceding clause, wherein the composition is composed of about 0.5% dexamethasone by total volume.

Clause 55: The method of reducing postoperative pain after a dental procedure of any preceding clause, wherein the composition is composed of about 0.05%-2% ketorolac tromethamine by total volume, more typically the composition is composed of about 0.5%-1.75% ketorolac tromethamine by total volume and even more preferred the composition is composed of about 1.25%-1.75% ketorolac tromethamine by total volume.

Clause 56: The method of reducing postoperative pain after a dental procedure of any preceding clause, wherein the composition is composed of about 1.5% ketorolac tromethamine by total volume.

Clause 57: The method of reducing postoperative pain after a dental procedure of any preceding clause, wherein the composition may include about 1-20 mg of dexamethasone, more typically about 4-15 mg of dexamethasone and even more preferred about 8-12 mg of dexamethasone.

Clause 58: The method of reducing postoperative pain after a dental procedure of any preceding clause, the composition may also include about 1-40 mg of ketorolac tromethamine, more typically about 10-35 mg of ketorolac tromethamine and even more preferred about 25-35 mg of ketorolac tromethamine.

Clause 59: The method of reducing postoperative pain after a dental procedure of any preceding clause, wherein the composition includes about 10 mg of dexamethasone.

Clause 60: The composition of any preceding clause, wherein the composition includes about 30 mg of ketorolac tromethamine.

1. A composition, comprising:
dexamethasone at a concentration of about 1-20 mg/ml; and
ketorolac tromethamine at a concentration of about 10-35 mg/ml.

2. The composition of claim 1, further comprising:
a local anesthetic.

3. The composition of claim 1, wherein the dry weight ratio of dexamethasone to ketorolac tromethamine in the composition is 1:2 to 1:4.

4. A kit sterilely received in a packaging, comprising:
a cartridge sterilely filled with a composition for use with a dental syringe, containing:
dexamethasone; and
ketorolac tromethamine.

5. The kit of claim 4, wherein the sterilely filled cartridge further contains a local anesthetic.

6. The kit of claim 4, wherein the cartridge includes a concentration of about 1-20 mg/ml of dexamethasone.

7. The kit of claim 4, wherein the cartridge includes a concentration of about 10-35 mg/ml of ketorolac tromethamine.

8. The kit of claim 4, wherein the composition includes about 10 mg/ml of dexamethasone.

9. The kit of claim 4, wherein the composition includes about 30 mg/ml of ketorolac tromethamine.

10. The kit of claim 4, further comprising:
a sterile dental syringe.

11. A method of reducing postoperative pain after a medical procedure, comprising:
administering a composition submucosally or subperiosteal via local infiltration, the composition comprising:
dexamethasone; and
ketorolac tromethamine.

12. The method of claim 11, further comprising:
adding a local anesthetic to the composition.

13. The method of reducing postoperative pain after a dental procedure of claim 11, wherein the composition is administered about 1 second to 60 minutes prior to the procedure, more typically about 1 minute to 30 minutes prior to the procedure and even more preferred less than about 5 minutes prior to the procedure.

14. The method of reducing postoperative pain after a dental procedure of claim 11, wherein the dexamethasone is a solution with at least about a 1-20 mg/mL concentration of dexamethasone, more typically about a 4-15 mg/mL concentration of dexamethasone and even more preferred about an 8-12 mg/mL concentration of dexamethasone.

15. The method of reducing postoperative pain after a dental procedure of claim 11, wherein the ketorolac tromethamine is a solution with at least about a 1-40 mg/mL concentration of ketorolac tromethamine, more typically about a 10-35 mg/mL concentration of ketorolac tromethamine and even more preferred about a 25-35 mg/mL concentration of ketorolac tromethamine.

16. The method of reducing postoperative pain after a dental procedure of claim 11, wherein the composition is composed of about 0.05%-1% dexamethasone by total volume, more typically the composition is composed of about 0.2%-0.75% dexamethasone by total volume and even more preferred the composition is composed of about 0.4%-0.6% dexamethasone by total volume.

17. The method of reducing postoperative pain after a dental procedure of claim 11, wherein the composition is composed of about 0.05%-2% ketorolac tromethamine by total volume, more typically the composition is composed of about 0.5%-1.75% ketorolac tromethamine by total volume and even more preferred the composition is composed of about 1.25%-1.75% ketorolac tromethamine by total volume.

18. The method of reducing postoperative pain after a dental procedure of claim 11, wherein the composition may include about 1-20 mg of dexamethasone, more typically about 4-15 mg of dexamethasone and even more preferred about 8-12 mg of dexamethasone.

19. The method of reducing postoperative pain after a dental procedure of claim 11, the composition may also include about 1-40 mg of ketorolac tromethamine, more typically about 10-35 mg of ketorolac tromethamine and even more preferred about 25-35 mg of ketorolac tromethamine.

20. The method of reducing postoperative pain after a dental procedure of claim 11, wherein the dry weight ratio of dexamethasone to ketorolac tromethamine in the composition is 1:2 to 1:4.
